(19) United States
(12) Patent Application Publication
(10) Pub. No.: US 2003/0177039 A1
(43) Pub. Date: Sep. 18, 2003

(54) Method of outsourcing IMRT services

(76) Inventors: Joseph A. Nicholas, Gibsonia, PA (US); Ronald Lalonde, Cranberry Twp., PA (US)

Correspondence Address: THORP REED & ARMSTRONG, LLP, ONE OXFORD CENTRE, 301 GRANT STREET, 14TH FLOOR, PITTSBURGH, PA, 15219-1425, US

(21) Appl. No.: 10/349,610

(22) Filed: Jan. 23, 2003

Related U.S. Application Data
(60) Provisional application No. 60/356,722, filed on Feb. 13, 2002.

(51) Int. Cl. A61N 5/10

(52) U.S. Cl. 705/2; 378/65

(57) ABSTRACT

A method of enabling a healthcare clinic to provide IMRT services on an outsourced basis comprises initially determining the existing capabilities of the clinic to deliver IMRT services. An implementation plan is developed based on the determining step. The implementation plan is designed to enable the clinic to achieve at least a baseline competence in delivering IMRT services. The implementation of the plan may include providing equipment, validating a communication link, providing policies and procedures for services to be rendered, and establishing a QA program for the services to be rendered. The clinic's staff is trained and tested. Thereafter, the readiness of the clinic to deliver IMRT services is evaluated based on the testing.

### BACKGROUND OF THE INVENTION

The present invention is directed to intensity modulated radiation therapy (IMRT) and, more particularly, to a method of setting up clinics to provide IMRT on an outsourced basis.

For most of the twentieth century, radiation treatment was administered by aiming imprecise, large, low-energy beams at the affected area. Tumor localization was by primitive radiological means or clinical palpation. Radiation biologists then established the biological principles of radiation treatment of most tumors, while setting dose parameters for normal tissue tolerance. Thus the need arose for conceptualizing and defining the parameters for radiation treatment planning rather than delivering empirical doses of radiation.

During the mid 1970's and early 1980's diagnostic imaging devices such as computed tomography (CT) scanners, primitive computers and linear accelerators were integrated to treat tumors with what is known today as 2-D treatment planning. For the first time, radiation oncologists and physicists were able to generate dose distributions based upon one, or at most a few, CT scan slices (images) to irradiate the tumor area, as opposed to only a single point dose calculation, the methodology commonly used in the preceding era. However, the limitations of 2-D treatment planning was clearly evident as it neglected to consider the volumetric shapes of both tumor and surrounding normal tissue, only measuring the contour on one small sample image. In the 2-D era, custom built lead-alloy blocks were inserted into the beams to physically limit the dose to the normal tissues. This method is still utilized in palliative terminal care such as symptomatic treatment of bone and brain metastasis. Unfortunately, it is also still the most common method used to shape the radiation field, both nationwide and indeed, worldwide.

By the late 1980's as computing technology advanced, 3-D treatment planning emerged as a more accurate method for planning the delivery of radiation. This process allowed tumors and normal tissues to be reconstructed and displayed in 3-D views, giving physicians and physicists the ability to aim multiple radiation beams at the target tumor, while minimizing radiation of normal tissue. Computerized devices were developed to scan the radiation beams and enter the data into treatment planning computers, allowing more accurate dose calculation algorithms to be utilized. Radiation oncologists could finally perform virtual simulation of a radiation beam hitting the target as seen from any linac gantry angle in space (“beam's-eye view”) and analyze the relationship of the beam aperture to the target volume and surrounding structures in near real time.

The 3-D beam's-eye view planning method projects a homogeneous cylinder of radiation over a 2-D compression of the tumor's shadow as seen by the beam from that direction. In the example of a kidney-shaped tumor, if the kidney “bean shape” is perpendicular to the central axis of the beam it will be recognized as kidney-shaped; if it is parallel to the beam's axis, it will be “seen” as a cylinder by the computer, completely missing the complex shaping. Conforming the dose distribution to this complex shape is limited by the fact that only multiple homogeneous beam intensities (or at most limited modulation using a wedge) are available with this technology.

Both 2-D and 3-D treatment planning paradigms are essentially trial and error methods in which computers are used to calculate radiation dose distributions from beam placement based on the clinical experience of the radiation oncologist, radiation physicist, and dosimetrist. Beam parameters including field size, beam weighting, beam angles and beam shapes are input in the computer and the resulting dose distribution is displayed; modifications on beam parameters are empirically applied and more dose distributions evaluated until the desired dose distribution is achieved. This method referred to as “forward planning” is time consuming, inefficient, and limits the number of beams to between two and six, because of the empiric nature of the process. These limitations result in dose distributions that do not optimally account for tumor target volume irregularities and do not optimally account for normal tissue location in relation to the target volume.

Another major advance in the implementation of 3-D treatment planning was the invention of the multi-leaf collimator (MLC). The MLC replaced the need to manufacture lead-alloy blocks used to shield normal tissues. It is mounted on a plastic tray, which is inserted into a slot in the linear accelerator's beam exit area. In the MLC, 80 to 120 computer-controlled leaves mimic the physical block. the MLC's ability to shape the periphery of the beam quickly and precisely is one of the primary reasons for the widespread introduction of conformal 3-D planning; MLC shapes are directly derived from treatment planning computers and sent to the linear accelerator in the form of computer files. The potential for MLC motion during radiation rather that just statically shaping the periphery of the beam heralded a long awaited solution for radiation treatment planning, i.e., true 3-D dose shaping or Intensity Modulated Radiation Therapy.

Intensity Modulated Radiation Therapy (IMRT) utilizes optimized, non-uniform radiation beam intensities incident on the patient, obtained from an inverse treatment planning system, and delivered with static MLC segments or dynamic multileaf motion during radiation. By creating three dimensional intensity profiles of the radiation beams and by assigning a different weight for each “beamlet”, the composite isodose curves will conform to the shape of the tumor volume, as well as avoiding critical structures. This process is in fact the first true 3-D radiation delivery methodology. The medical affect of IMRT is enormous; higher cure rates (as more radiation can be delivered into the tumor tissues where it is most needed) and less normal tissue complications (as less high-dose radiation is delivered to normal tissues). This ability to precisely shape and increase or decrease the strength of the radiation beam according to tumor size, shape and metabolic activity is unprecedented in the history of radiation oncology and is a major advance for multi-disciplinary cancer care.

The complexity of true 3-D radiation dose calculations makes IMRT planning by the forward method a near impossible undertaking. Thus, IMRT necessitated the creation of inverse treatment planning computer algorithms and programs. Instead of empirically pre-setting beam directions, weights and shapes and then computing the resulting dose distributions as in 3-D forward planning, IMRT reverses the process. Physicians and physicists outline their objectives in terms of radiation dose distribution by making mathematical statements of the dose volume constraints that are then translated into the beam configurations that will deliver the tumor and normal tissue dose-volume histograms prescribed. It is a complex computing process that results in dose optimization at its highest level.

IMRT was initially delivered through multiple static portals of entry; MLC leaves moved quickly to certain positions, the radiation beam went on and off and this process was repeated dozens to hundreds of times (referred to as segmental-MLC (SMLC) or “step-and-shoot” technique. In some instances, 3-D dose shaping with the SMLC method may not be optimal and treatment delivery times prolonged. Better inverse treatment planning computer power and automatic multileaf sequencers yielded the dynamic-MLC (DMLC) or “sliding window” technique, where continuous motion of the MLC leaves flow throughout the entire radiation treatment sequence, while the linear accelerator rotates around a patient precisely placed at a preset center point.

In the last five years, there have been innumerable clinical studies showing the efficacy, safety and superiority of IMRT as compared to 3-D conformal and other means of radiation delivery. All of these confirm what is easily seen when looking at computer generated IMRT radiation dose distributions: dose to tumor or target tissues at risk is equal or higher while high dose to normal tissues is significantly reduced.

Secondary to its complexity, costs associated with IMRT treatment are higher that 3-D conformal radiation. For proper IMRT planning and delivery, the linear accelerator needs to be state of the art, i.e. digitally controlled and outfitted with a multileaf collimator. An inverse treatment planning computer system needs to be purchased and commissioned. The beams have to be studied in terms of photon intensity profiles rather than plain portal films. The physicians, physicists, dosimetrists, and therapists have to be familiar with all the above technical updates. The physician has to spend significantly more time in contouring targets and normal structures, evaluating the resulting plan, as well as prescribing the radiation treatment in terms of desired dose volume histograms rather that doses alone. For many clinics, the cost of obtaining the equipment and hiring the necessary personnel is prohibitive. For others, the patient volume may not justify such expenditures. Thus, the need exists for a method of enabling such clinics to provide life saving IMRT.

### BRIEF SUMMARY OF THE INVENTION

The present invention is directed to a method comprising initially determining the existing capabilities of a healthcare clinic to deliver IMRT services. An implementation plan is developed based on the determining step. The implementation plan is designed to enable the clinic to achieve at least a baseline competence in delivering IMRT services. The implementation of the plan may include providing equipment, validating a communication link, providing policies and procedures for services to be rendered, and establishing a QA program for the services to be rendered. The clinic's staff is trained and tested. Thereafter, the readiness of the clinic to deliver IMRT services is evaluated based on the testing. This process enable the clinic to deliver IMRT services on an outsourced basis.

The method of the present invention provides a number of advantages including the rapid implementation of a leading-edge IMRT program via outsourced support for training, treatment planning and quality assurance. The present invention provides a turnkey solution that enables clinics to deliver IMRT with existing clinical personnel thereby providing improved clinical benefits for patients, enhanced revenue for the clinic and decreased startup costs related to technology and staffing requirements. Those advantages and benefits, and others, will be apparent from the Detailed Description of the Invention below.

### BRIEF DESCRIPTION OF THE DRAWINGS

For the present invention to be easily understood and readily practiced, the present invention will now be described for purposes of illustration and not limitation in connection with the following figures in which:

FIG. 1 is a block diagram of the system and method of the present invention;

FIGS. 2 and 3 illustrate sample forms which may be used to assess equipment at a clinic;

FIG. 4 illustrates a sample form which may be used to assess the knowledge level and skill set of a clinical team; and

FIG. 5 is a block diagram of consultation services which may be provided to the clinic from a central location.

### DETAILED DESCRIPTION OF THE INVENTION

The present invention provides rapid implementation and ongoing support of IMRT programs via outsourcing of support for training, treatment planning and quality assurance. This enables a clinic having a shortage of clinic resources and of IMRT knowledge to provide IMRT with leading standards without adding expensive staff.

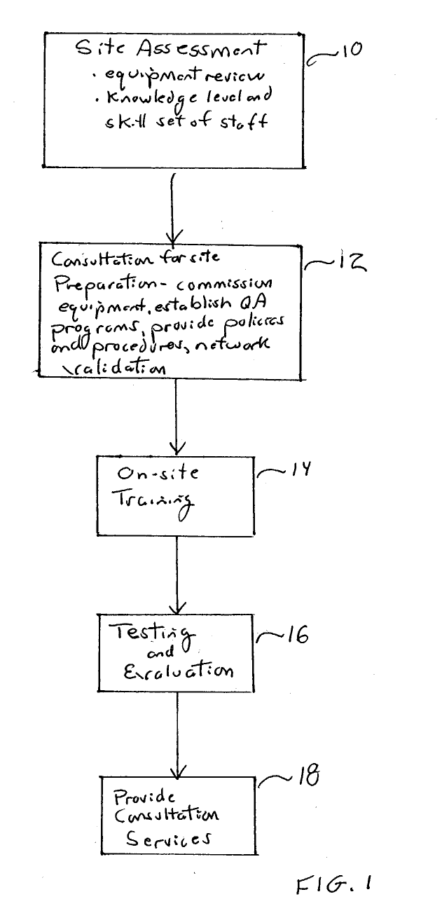

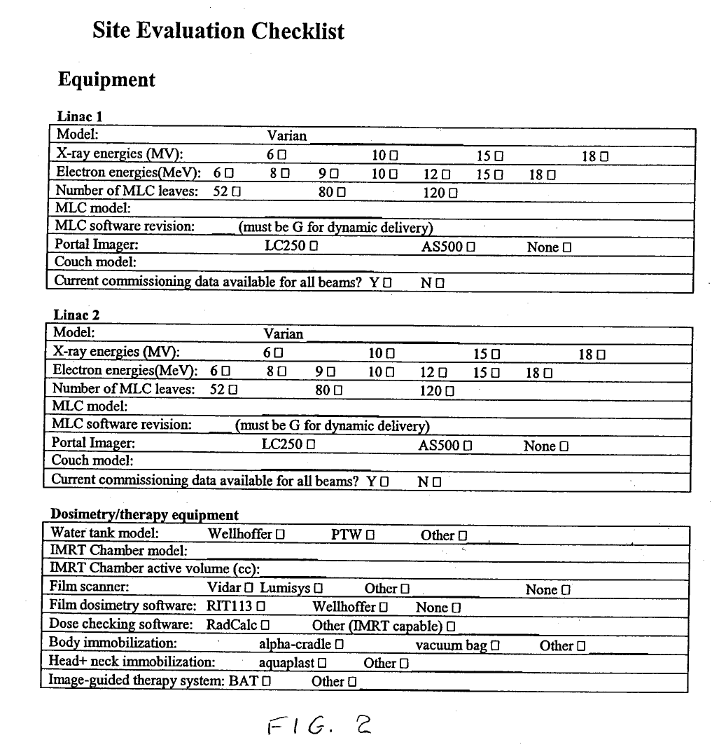

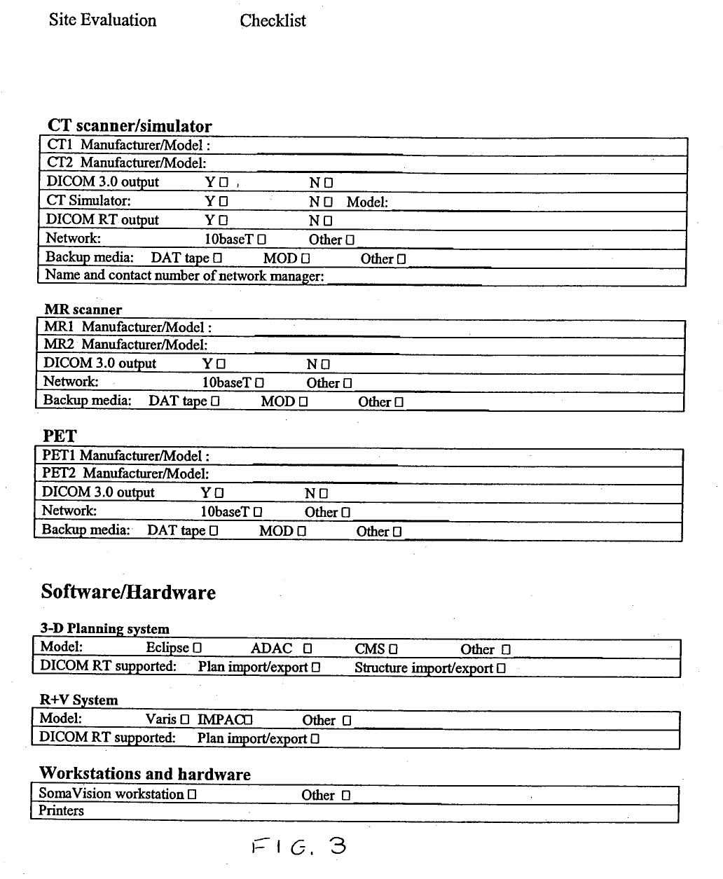

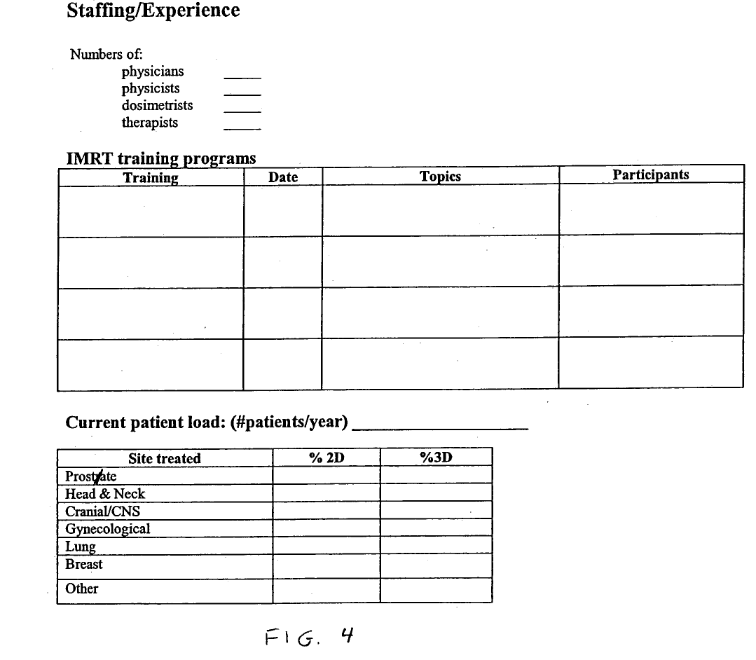

Prior to beginning the remote treatment planning process, the present invention contemplates a site assessment as shown by block 10 in FIG. 1. The site assessment includes a number of items. Utilizing a set of established forms and a baseline set of capabilities, a determination is made regarding the existing capabilities of the site from a clinical and technical standpoint. Using, for example, the forms shown in FIGS. 2 and 3, an inventory of the existing equipment and technology including hardware, networking capabilities, other equipment and software is prepared. Using, for example, the form in FIG. 4, the knowledge level and skill set of the clinical team (physicians, physicists, dosimetrists, and technicians) regarding radiation therapy and IMRT is assessed. Those of ordinary skill in the art will recognize that the type of forms used to capture the information from the site evaluation can vary such that the present invention is not limited to the forms shown in FIGS. 2, 3, and 4.

Comparing the findings of the site assessment to a baseline set of capabilities, an implementation plan is customized for the site. Implementation may include:

consultation for site preparation,

commissioning of radiation delivery equipment and treatment planning system,

network validation,

providing the clinic with policies and procedures, and

establishment of a QA program.

That phase is represented in FIG. 1 by block 12. Implementation is the first step toward raising the existing level of capabilities of the clinic to at least the baseline capabilities.

An example of a commissioning procedure for radiation delivery equipment follows. Clearly, the commissioning procedure will vary depending on the type of equipment being commissioned. However, the purpose of the commissioning procedure is to insure that the equipment has been properly set up. The following procedure is exemplary only and not intended to limit the present invention.

Subject: Entry and Verification of Linac Beam Data for Treatment Planning

1. Use the appropriate software to generate CADPLAN format data files (use Wellhoffer or PTW filters). The following files are necessary for photon beam, IMRT-only commissioning:

CDP file containing PDD scans, square field sizes from 3×3-40×40

CDP file containing crossplane profile scans, same field sizes, 5 depths=dmax, 5, 10, 20, 30 cm.

CDP file containing diagonal scans of 40×40 field, depths=dmax, 5, 10, 20, 30 cm.

Excel spreadsheet containing output factors for square fields and relative output factors for rectangular fields.

2. Enter the beam data into CADPLAN. Print out all configuration information, and plot out all raw and converted beam data. Follow steps as described in CADPLAN beam modeling manual to complete commissioning.

3. Use plan document/users/patients/imrt/acceptance phantom to generate test plans for square and rectangular fields. Compare plan dose to commissioning measurements according to the criteria given in the document. Print out the completed document and store one copy with beam plots. Print a second copy for the site.

4. Have the site CT scan their validation phantom and send the scans to CADPLAN. Using the new beam model, generate at least 4 standard IMRT plans, (at least 2 for each energy if a dual-energy machine) as contained in the/users/patients/imrt directory. The plans should cover at least 2 different treatment sites.

5. Deliver (or have the site deliver) plans created in step 4 to phantom. Confirm point dose with chamber, confirm 2-dimensional dose distribution using EDR film. Use RIT software to measure absolute dose on film and create a plan/film isodose plot for each plan.

6. Create a plan validation report for each validation plan, and store one copy with the beam plots, and deliver one copy to the site. Store the films with the validation documents.

An example of a network validation procedure for a communications network follows. As with the commissioning procedure, the purpose of the validation procedure is to insure that the data transfer between the clinic and the central site is working properly. The following procedure is exemplary only and not intended to limit the present invention.

Subject: Network Validation

1. Validation of data transfer across network connections will take place after initial installation and verification of the network line by the telecommunications vendor.

2. A standard Somavision patient data set will be transmitted from the central server consisting of: i) A series of CT images of a CT density phantom; ii) a volumetric CT set reconstructed from these images; iii) a series of tissue contours of fixed size and shape; iv) a treatment plan consisting of treatment beams of fixed sizes in a fixed geometrical arrangement.

3. The client site physicist/dosimetrist will review the following information for accuracy and completeness:

patient name and ID

number of CT slices

size of phantom

number and position of structure contours

size of structure contours

CT number of phantom inclusions

number and arrangement of treatment beams

treatment machine ID

4. The client site will then resend the data set to the central server, using DICOM RT transfer. The physicist/dosimetrist at the central site will verify the accuracy and completeness of all information as given above.

5. In addition, cyclic redundancy checksums will be calculated for a series of files transmitted to the site. The site will then retransmit the information to the central server as before. Upon return, the cyclic redundancy checksums will be recalculated to ensure that no data was corrupted or lost in the transfer.

The provision of policies and procedures is to document the proper way each aspect of operation of the clinic and every type of service provided by the clinic should be performed. For example, a policy and procedure for the validation of IMRT plans prior to delivery follows. The following procedure is exemplary only and not intended to limit the present invention.

Subject: Validation of IMRT Plans Prior to Delivery

1. If the customer has access to a dose calculation engine (CADPLAN or Eclipse) the generation of the phantom plan may be performed at the customer's location. If the customer does not have access to dose calculation, the phantom plan will be generated at the central service location and delivered to the customer.

2. The customer will perform plan validation for at least the initial 5 patients treated at each anatomical site.

3. At the customer's linear accelerator, set up the phantom exactly as it was set up when CT scanned for treatment planning. If film is to be used with the phantom, for example, it is recommended that the phantom be scanned with film(s) placed at the customer's location that will be used for plan validation.

4. Mark the film with a pin 10 cm away from the field center on the +−X, +−Y axes. The pin marks serve to confirm isocenter and scale when scanning the film using film dosimetry software, and may be used to register planning system isodose plots to the film.

5. Using the patient MLC delivery files, deliver the plan to the phantom, using chamber and film to record dose. For long, high MU deliveries it is recommended that the customer determine leakage reading from the chamber/electrometer pair used for phantom validation, and account for this in final dose calculation.

6. After delivering the plan, shift the film so that one corner of the film, well away from the IMRT plan, is centered in the beam, with the chamber still at isocenter, and deliver a 5×5 field, 200 MU, to the film and to the chamber. Record the chamber readings. Develop the film and use film dosimetry software to determine dose to the film center and to the center of the 5×5 field. The 5×5 field will be used to correct the calibration for this film. Use film dosimetry software to compare isodose distribution between the plan and the film. Recommended acceptance criteria for absolute dose is within 5% of plan, isodose lines within 5% or 4 mm of plan.

7. Use the 5×5 chamber reading to correct the plan chamber reading for daily variation of linac output, and make corrections for leakage if necessary. Determine absolute dose to the chamber. Recommended acceptance criteria for absolute dose is within 5% of plan prediction for the chamber active volume.

8. If either chamber or film does not pass the stated validation criteria, it may be necessary to shift the phantom so that the chamber and film measurement locations are within a more uniform region of the dose. Repeat the validation as necessary until acceptance criteria are met. If the absolute dose acceptance criteria cannot be met, but the 2-dimensional relative dose distribution is acceptable, adjust the MU in the plan to bring the absolute dose into acceptable range, and make a note on the patient's plan describing this change. If the absolute dose from the chamber or the relative dose from the film do not pass acceptance criteria, the IMRT plan may not be used for treatment.

9. Create a validation report for the patient's plan. Sign and date the validation and put in the patient's chart. Attach the plan/film isodose plot to the report.

Finally, the establishment of a QA program may be accomplished by providing the clinic with additional policies and procedures directed to quality assurance issues. Providing a series of QA protocols that are effective, yet efficient, decreases the time requirements for a clinic's staff on a per patient basis. An example of a QA policy and procedure to ensure that treatment delivery is in accordance with the written directive (prescribed treatment) and plan of treatment, and is in compliance with AAPM Task Group #40 Recommendations on Comprehensive QA for Radiation Oncology follows. A group of such policies and procedures is necessary for every aspect of operation and every type of service provided by the clinic. The following procedure is exemplary only and not intended to limit the present invention.

Subject: Quality Assurance Verification of Treatment Data

1. The radiation oncologist shall verify the anatomic location visually as well as radiographically during simulation.

2. Treatment fields will be approved only after a review of treatment information relative to the site of treatment and the plan of treatment are performed.

3. Treatment Site: entails a comparison of the treatment prescription to anatomic location by review of simulation and verification films, review of portals directed to the site, and review of blocking patterns for each treatment portal.

4. Treatment Plan: entails review of daily fraction dose, number of fractions, arrangement of treatment portals, beam modifying devices.

5. It is the radiation therapist's responsibility to verify that the anatomic placement of treatment matches the prescribed site of treatment prior to treatment delivery.

Simulation

A. The radiation oncologist is responsible for verification of the anatomic location of the intended treatment portals:

Physically on the patient (visual confirmation)

Radiographically (radiographs of treatment set-up port and treatment portals).

The physician's signature/initials and date on the radiographs shall indicate approval of the treatment ports.

B. It is the responsibility of the therapist performing simulation to:

Provide the radiation oncologist with radiographs that demonstrate the area of interest adequately (proper radiographic technique, appropriate film placement)

Demonstrate direction and anatomic placement of the field radiographically by use of lead letter markers (RT LAT, RAO, POST L) and graticule

Ensure that all simulation radiographs are signed/dated by the radiation oncologist

Provide complete documentation of the simulated treatment parameters and patient setup on the Technical Instruction Sheet (TIS), initialing the TIS to indicate this review.

Provide diagram of the setup portal and/or treatment portals.

Photograph(s) of treatment ports can be used and are required on all electron field simulations and simulations that necessitated non-standard patient position or setup technique.

C. Radiographs/charts which do not meet with criteria above will be returned to the responsible therapist for completion (no calculation and/or no approval will be provided).

Verification

A. It is the responsibility of the designated therapist performing verification to:

Physically enter the treatment room with the treatment record, observe the setup for anatomic accuracy against the written prescription, and observe and verify that the setup matches the TIS parameters.

Obtain portal verification films of ALL treatment fields. All films must have lead markers indicating direction/anatomic placement of the treatment field (AR, PL, R LAT, MED L) and graticule to indicate CAX/field size. All films must be labeled with patient's name (last/first), date, and field title.

Compare all SSD's against simulated values, and document changes in SSD/separation on the TIS.

Ensure all gantry/collimator angles are correct according to treatment plan (simulation)

Ensure all treatment data is correct and consistent between the written TIS and R+V.

Document all information in ink.

B. The therapist responsible for the verification will initial the TIS indicating completion of checks of all treatment data.

C. Completed verification port films will be directed to the radiation oncologist for review/approval.

D. Port films/technical instructions that do not meet the stated criteria may not be calculated/approved.

E. n the event that the criteria indicated in A, B, or C cannot be met, but treatment is indicated, the ordering or on-call MD should be paged to received verbal approval to proceed. This should be documented in the treatment record.

Treatment Field Approval

It is the responsibility of the individual providing field approval to:

A. Compare the anatomic location of the prescribed (written) course of therapy to the simulation and verification films. Compare MLC blocking patterns—R+V to intended (simulated). Approval may not be provided for treatment fields without approved verification films, or with incomplete instructions.

B. Compare the number and direction of portals directed to the site, and ensure the gantry angle(s) match the treatment plan and TIS.

C. Compare calculated MU's/daily dose between calculation sheet/treatment record/R+V.

D. Check Wedge/Orientation—planned to diagrammed to R+V.

Daily Treatment Delivery

A. It is the responsibility of the designated therapist operating the treatment unit console to:

Physically enter the treatment room with the treatment record, observe the setup for anatomic accuracy, and observe and verify that the setup matches the TIS parameters.

Compare treatment parameters in chart to R+V for each treatment portal prior to delivery.

Document treatment administration in the treatment record.

B. It is the responsibility of the designated therapist to indicate boost planning, field size/SSD/separation changes on the TIS, tag the chart with a “Physics Request”, and forward to Dosimetry.

The next phase in the process, represented by block 14 in FIG. 1, is on-site training (didactic and hands on) for the entire clinical team. The training program may include:

an introduction of IMRT, CT scanning and simulation,

contouring,

exporting and importing plans,

interfacing with record and verify systems,

plan delivery,

dose validation,

patient selection,

immobilization and organ motion,

dose prescriptions,

plan evaluation,

patient education,

plan delivery, and

QA.

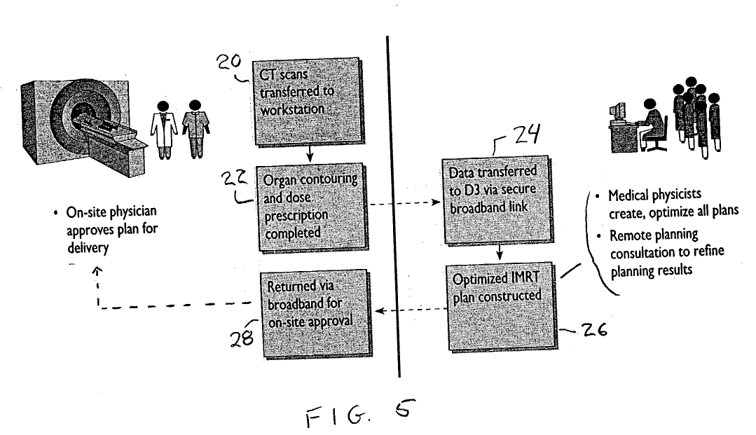

After the on-site training phase, a testing and evaluation phase, represented by block 16 in FIG. 1 is implemented. The purpose of the testing and evaluation phase is to insure that the clinic is ready to deliver IMRT services. Once the clinic is deemed ready to proceed, the remote treatment planning process is initiated and the site begins delivering IMRT. After the program is established, a range of consultation services to promote a successful IMRT program at the clinic may be provided as represented by the block 18 in FIG. 1, which is shown in detail in FIG. 5.

Referring to FIG. 5, a clinician at the clinic site loads the CT scan into a work station at step 20 and performs contouring, sets goals, and prescribes doses thereby producing pre-planning data at step 22. The data is then transmitted via a secure connection to a central office as shown by step 24.

At the central office, a member of a physics team downloads the pre-planning data. The data is reviewed for quality and completeness according to established standards and protocols for planning. Should a team member determine that pre-planning data requires revision to obtain optimal results, the site physicist is contacted and a remote consultation occurs. Through that process, the local physicist learns the essentials of treatment planning. Should an issue exist with data supplied by the physician, the physicist at the central location will engage a consulting radiation oncologist, as necessary, to work directly with the clinic physician to consult.

Once agreement is reached regarding the pre-planning data, the central location physics staff creates an optimized IMRT treatment plan as shown by step 26 and returns it via the secure connection to the clinic at step 28.

Upon its return, the optimized IMRT plan is reviewed by the site physicist and physician and accepted/approved for delivery. After acceptance local QA is performed at the site and IMRT treatment is delivered to the patient.

While the present invention has been described in conjunction with preferred embodiments thereof, those of ordinary skill in the art will recognize that many modifications and variations are possible. For example, the forms used to capture information from the clinic assessment may vary substantially from the forms shown in the figures. The policies and procedures will vary depending upon the types of services to be delivered and the baseline quality believed to be necessary for that service. The present invention is not intended to be limited by the specific forms and procedures disclosed herein, but only by the scope of the following claims.

What is claimed is:

1. A method, comprising:
determining the existing capabilities of a healthcare clinic to deliver IMRT services;
developing an implementation plan based on said determining step;
implementing the implementation plan including providing equipment, validating a communication link, providing policies and procedures for services to be rendered, and establishing a QA program for the services to be rendered;
providing training to the clinic's staff;
testing the clinic's staff; and
evaluating the readiness of the clinic to deliver IMRT services based on said testing.

2. The method of claim 1 wherein said step of providing equipment includes providing and commissioning radiation delivery equipment.

3. The method of claim 1 additionally comprising providing consulting services to said healthcare clinic.

4. A method, comprising:
determining the existing capabilities of a healthcare clinic to deliver IMRT services;
providing equipment, policies and procedures for services to be rendered in response to said determining step;
validating a communication link;
providing training to the clinic's staff,
testing the clinic's staff; and
evaluating the readiness of the clinic to deliver IMRT services based on said testing.

5. The method of claim 4 wherein said providing policies and procedures includes providing QA policies and procedures.

6. The method of claim 5 wherein said step of providing equipment includes providing and commissioning radiation delivery equipment.

7. The method of claim 4 additionally comprising providing consulting services to said healthcare clinic.

8. A method, comprising:
evaluating the existing capabilities of a healthcare clinic to deliver IMRT services;
providing and commissioning equipment based on said evaluating step;
providing policies and procedures for services to be rendered based on said evaluating step;
validating a communication link; and
training the clinic's staff.

9. The method of claim 8 wherein said providing policies and procedures includes providing QA policies and procedures.

10. The method of claim 8 wherein said step of providing and commissioning equipment includes providing and commissioning radiation delivery equipment.

11. The method of claim 8 additionally comprising providing consulting services to said healthcare clinic.

12. The method of claim 8 additionally comprising evaluating the readiness of the clinic to deliver IMRT services.

13. The method of claim 12 wherein said evaluating the readiness includes testing the clinic's staff.

14. A method comprising:
evaluating the existing capabilities of a healthcare clinic to deliver IMRT services;
providing training and equipment to raise the level of the clinics capabilities to deliver IMRT services to at least a baseline capability; and
providing consulting services from a remote location to the clinic to support the clinic's delivery of IMRT services.

15. The method of claim 14 wherein said providing equipment includes providing and commissioning radiation delivery equipment.

16. The method of claim 14 wherein said providing equipment includes commissioning a communication link.

17. The method of claim 14 additionally comprising providing policies and procedures for services to be rendered.

18. The method of claim 17 wherein said policies and procedures include QA policies and procedures.

19. The method of claim 14 additionally comprising training the clinic's staff and testing the clinic's staff.

20. The method of claim 19 additionally comprising evaluating the readiness of the clinic to deliver IMRT services based on said testing.
